(19) United States
(12) Patent Application Publication
(10) Pub. No.: US 2026/0102497 A1
(43) Pub. Date: Apr. 16, 2026

(54) PHOTOSENSITIVE HYBRID VESICLES AND THE FABRICATION METHODS AND APPLICATIONS THEREOF

(71) Applicant: The University of Hong Kong, Hong Kong (HK)

(72) Inventors: Wenwei TU, Hong Kong (HK); Yifan GAO, Hong Kong (HK); Jinzhao LIU, Hong Kong (HK); Weiping WANG, Hong Kong (HK)

(21) Appl. No.: 19/348,753

(22) Filed: Oct. 2, 2025

Related U.S. Application Data
(60) Provisional application No. 63/708,243, filed on Oct. 16, 2024.

(51) Int. Cl. A61K 41/00; A61K 9/127; A61K 9/1277; A61K 31/7105; A61K 35/768; A61N 5/06

(52) U.S. Cl. CPC A61K 41/0071; A61K 9/127; A61K 9/1277; A61K 31/7105; A61K 35/768; A61N 5/062; A61N 2005/0663

(57) ABSTRACT

A photosensitive hybrid vesicle is provided, including a photosensitizer-loaded liposome fused with a γδ-T exosome via a membrane fusion process. The resulting hybrid vesicle integrates the structural and functional characteristics of both components, retaining functional surface proteins and cytolytic molecules derived from the γδ-T exosome while incorporating the photosensitizer from the liposome. Upon exposure to light, the hybrid vesicle generates reactive oxygen species (ROS) for enhanced therapeutic activity, enabling combined exosome-mediated cytolytic effects and photosensitizer-mediated photodynamic therapy in a single nanoscale delivery system.

### CROSS-REFERENCE TO RELATED APPLICATIONS

The present application claims priority from U.S. Provisional Utility Patent application No. 63/708,243 filed Oct. 16, 2024; the disclosure of which is incorporated herein by reference in its entirety.

### FIELD OF THE INVENTION

The present invention generally relates to nanomedicine and immunotherapy, particularly to bioengineered vesicles that combine features of immune cell-derived exosomes and photosensitizer-loaded liposomes. More specifically, the present invention relates to photosensitive hybrid vesicles formed by membrane fusion of γδ-T cell-derived exosomes with photosensitizer-loaded liposomes for targeted cancer therapy.

### BACKGROUND OF THE INVENTION

Melanoma is among the most aggressive forms of skin cancer, characterized by high metastatic potential and poor prognosis. With only a few standard treatment options available, the median survival time is typically between three and eleven months. Conventional therapies for melanoma, such as chemotherapy and radiotherapy, lack tumor-specific targeting and provide only short-term protection against relapse. This has driven significant interest in developing targeted therapies to improve treatment outcomes and long-term prognosis.

Photodynamic therapy (PDT) has emerged as a minimally invasive therapeutic approach suitable for melanoma due to its spatiotemporal control. PDT begins when a photosensitizer absorbs photon energy and transitions from the ground state to the excited singlet state. From there, it either decays back to the ground state via fluorescence emission or undergoes intersystem crossing to the triplet state. In the triplet state, the photosensitizer interacts with molecular oxygen and other endogenous substrates to generate reactive oxygen species (ROS) such as singlet oxygen (¹O₂), hydrogen peroxide (H₂O₂), and superoxide (O₂·⁻). When laser irradiation is locally applied to the lesion, accumulated photosensitizer molecules are activated to eradicate tumor tissue via ROS generation without damaging surrounding normal tissue. Furthermore, PDT can induce immunogenic cell death (ICD), which stimulates both innate and adaptive immune responses for long-term antitumor immunity. This process typically involves the release or surface exposure of damage-associated molecular patterns (DAMPs) such as calreticulin (CRT), high-mobility group box 1 (HMGB1), and ATP from tumor cells. DAMP presentation promotes the maturation of antigen-presenting cells (APCs), enabling them to present tumor antigens and activate tumor-specific T-cell responses.

To deliver photosensitizers to tumor tissues effectively, nanoparticle formulations with high drug-loading capacity and improved pharmacokinetics are frequently used. However, conventional nanoscale carriers such as polymeric micelles, liposomes, and lipid nanoparticles have several drawbacks, including rapid immune clearance, excessive liver accumulation, and relatively low biocompatibility.

Human γδ-T cells represent a small but functionally distinct subset of T lymphocytes, comprising about 1-10% of total T cells in peripheral blood and lymphoid tissues. Based on differences in their T-cell receptor (TCR) δ chains, γδ-T cells can be divided into Vδ1 and Vδ2 subpopulations. Among these, Vγ9Vδ2-T cells are predominant in peripheral blood and lymphoid tissue and can be activated in an MHC-independent manner by phosphoantigens, which are nonpeptidic phosphorylated intermediates of the mevalonate pathway. Previous studies have shown that pamidronate (PAM), a pharmacological aminobisphosphonate used for osteoporosis, induces the accumulation of isopentenyl pyrophosphate (IPP) in mammalian cells, thereby stimulating the proliferation and cytotoxic activity of Vγ9Vδ2-T cells both in vitro and in vivo. Activation of γδ-T cells by PAM has been demonstrated to effectively treat Epstein-Barr Virus (EBV)-induced B-cell cancers in humanized mouse models. However, the therapeutic efficacy of PAM-expanded γδ-T cells against solid tumors remains limited due to the immunosuppressive tumor microenvironment and poor infiltration of γδ-T cells into solid tumors. These limitations have motivated the exploration of cell-free therapeutic approaches, such as using extracellular vesicles derived from γδ-T cells, to broaden clinical applications and improve therapeutic outcomes in solid tumor treatment.

Exosomes—small extracellular vesicles ranging from 20-200 nm—originate from endosomes and mediate intercellular communication by transferring lipids, proteins, and nucleic acids. Unlike artificial nanoparticles, exosomes naturally inherit functional molecules from their parental cells, conferring high biocompatibility and improved stability during circulation. Immune cell-derived exosomes, such as those from neutrophils, macrophages, and dendritic cells, have already shown great promise in cancer immunotherapy. More recently, exosomes derived from human γδ-T cells (γδ-T exosomes) have demonstrated the ability to control EBV-associated tumors by directly inducing cancer cell apoptosis and enhancing antitumor αβ-T cell responses. In addition to EBV-induced B-cell cancers, γδ-T exosomes also show therapeutic effects against solid tumors, including EBV-associated gastric carcinoma and nasopharyngeal carcinoma (EBV-NPC). Notably, γδ-T exosomes can also be used as tumor vaccines when conjugated with tumor-associated antigens, simultaneously killing tumor cells and inducing tumor-specific αβ-T cell responses. Compared with adoptive cell therapy, cell-free γδ-T exosomes offer several advantages: they are resistant to the tumor immunosuppressive microenvironment, exhibit higher clinical safety, allow for scalable production at lower cost, and maintain long-term stability during storage, underscoring their strong translational potential.

Despite these promising findings, it remains unclear whether γδ-T exosomes can also be applied to highly malignant cancers such as melanoma. The limited efficacy of PAM-expanded γδ-T cells against solid tumors—due to the immunosuppressive tumor microenvironment and poor infiltration—highlights an urgent need for innovative approaches. Accordingly, the present invention aims to overcome these obstacles and expand the therapeutic potential of γδ-T exosomes to treat high-malignancy cancers such as melanoma.

### SUMMARY OF THE INVENTION

It is an objective of the present invention to provide the compounds, compositions, and methods to solve the aforementioned technical problems.

In accordance with a first aspect of the present invention, a photosensitive hybrid vesicle is provided. The photosensitive hybrid vesicle includes a photosensitizer-loaded liposome and a γδ-T exosome. Specifically, the γδ-T exosome and the photosensitizer-loaded liposome are fused together via membrane fusion.

In accordance with one embodiment, the photosensitive hybrid vesicle includes a lipid bilayer structure.

In accordance with another embodiment, the photosensitive hybrid vesicle retains functional surface proteins and cytolytic molecules from the γδ-T exosome and incorporates the photosensitizer from the photosensitizer-loaded liposome for generating ROS upon light irradiation.

In accordance with yet another embodiment, the cytolytic molecules include granzyme A, granzyme B, perforin and granulysin; and the functional surface proteins include PD-1 and CCR5.

In accordance with yet another embodiment, the photosensitive hybrid vesicle further includes a therapeutic agent, including a chemotherapeutic agent, a monoclonal antibody, an immune checkpoint inhibitor, an immunomodulatory cytokine, an siRNA or miRNA, a gene-editing cargo, a radiotherapeutic agent, a targeted small-molecule inhibitor, and an oncolytic virus.

In accordance with yet another embodiment, the photosensitizer includes chlorin e6 (Ce6), a porphyrin, phthalocyanine, a bacteriochlorin derivative, or a combination thereof.

In accordance with yet another embodiment, the vesicle is formulated to deliver hydrophobic drugs, hydrophilic drugs, or both simultaneously.

In accordance with yet another embodiment, the membrane fusion is achieved by a freeze-thaw process, polyethylene glycol-mediated fusion, natural incubation, or extrusion-based fusion.

In accordance with yet another embodiment, the vesicle exhibits an average particle size between 50 nm and 300 nm and maintains structural stability under physiological conditions for at least 48 hours.

In accordance with yet another embodiment, the vesicle further includes targeting ligands or peptides to enhance tissue or tumor specificity.

In accordance with yet another embodiment, the vesicle further includes one or more targeting ligands selected from antibodies, antibody fragments, peptides, aptamers, or small molecules that specifically bind to a tumor-associated antigen or receptor.

In accordance with a second aspect of the present invention, a pharmaceutical composition is provided. Particularly, the pharmaceutical composition includes the aforementioned photosensitive hybrid vesicle and a pharmaceutically acceptable addition.

In accordance with one embodiment, the pharmaceutically acceptable addition comprises one or more of an excipient, a stability additive, a carrier, a diluent, or a solubilizer.

In accordance with a third aspect of the present invention, a method of treating a cancer in a subject is provided. Specifically, the method includes:

administering a therapeutically effective amount of the aforementioned pharmaceutical composition to the subject; and exposing a tumor tissue area of the subject to light of a wavelength to activate the photosensitive hybrid vesicle.

In accordance with one embodiment, the cancer includes melanoma, retinoblastoma, colon cancer, breast cancer, gastric cancer, pancreatic cancer, lung cancer, and any solid tumors.

In accordance with another embodiment, the wavelength is between 630 nm and 680 nm.

In accordance with yet another embodiment, the pharmaceutical composition is administered intravenously, intratumorally, or topically.

In accordance with a fourth aspect of the present invention, a method of manufacturing the aforementioned photosensitive hybrid vesicle is disclosed. Particularly, the method includes the following steps: preparing a photosensitizer-loaded liposome; isolating a γδ-T exosome from γδ-T cells; fusing the photosensitizer-loaded liposome and the γδ-T exosome via a membrane fusion technique; and obtaining a photosensitive hybrid vesicle. Resultingly, the photosensitive hybrid vesicle retains functional surface proteins and cytolytic molecules from the γδ-T exosome and incorporates the photosensitizer from the photosensitizer-loaded liposome for generating ROS upon light irradiation.

In accordance with one embodiment, the cytolytic molecules comprise granzyme A, granzyme B, perforin and granulysin; and the functional surface proteins comprise PD-1 and CCR5.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

Embodiments of the invention are described in more details hereinafter with reference to the drawings, in which:

FIG. 1 depicts a schematic of photosensitive hybrid vesicles preparation;

FIGS. 2A-2N depict the preparation and characterization of photosensitive hybrid γδ-T exosomes, in which: FIG. 2A shows the characterization of membrane fusion between liposomes and γδ-T exosomes by flowcytometric small particle detection; FIG. 2B is a schematic illustration of the membrane fusion between liposomes and γδ-T exosomes and the increased distance between FRET pairs after fusion; FIG. 2C depicts the fluorescence spectra of Rhodamine B and Ce6; FIG. 2D depicts the fluorescence spectra change of hybrid vesicles with Rhodamine B and Ce6 loaded before and after membrane fusion process; FIG. 2E shows the transmission electron microscopy (TEM) images of Ce6-loaded liposomes, γδ-T exosomes, and hybrid vesicles; FIG. 2F, FIG. 2G and FIG. 2H respectively show the size distribution of Ce6-loaded liposomes (FIG. 2F), γδ-T exosomes (FIG. 2G), and hybrid vesicles (FIG. 2H); FIG. 2I depicts the zeta potential of Ce6-loaded liposomes, γδ-T exosomes, and hybrid vesicles; FIG. 2J depicts the stability test of hybrid vesicles under physiological conditions; FIG. 2K shows the UV-Vis absorption spectra of free Ce6 and hybrid vesicles in the presence of PBS buffer; FIG. 2L depicts the SDS-PAGE analysis of Ce6-loaded liposomes, γδ-T exosomes, and hybrid vesicles; and FIG. 2M and FIG. 2N show the characterization of the surface markers of γδ-T exosomes (FIG. 2M) and hybrid vesicles (FIG. 2N) by flow cytometry;

FIG. 3 depicts the fluorescence excitation spectrum of Ce6;

FIGS. 4A-4I depict the active targeting effect of hybrid γδ-T exosomes to promote tumor accumulation, in which: FIG. 4A and FIG. 4B show the cellular uptake of Ce6 in A375 cells treated with PBS, free Ce6, Ce6-loaded liposomes, and hybrid γδ-T exosomes; FIG. 4C depicts the representative images of CLSM examination of Ce6 uptake inside A375 cells with different formulations; FIG. 4D and FIG. 4E show the flow cytometry measurement (FIG. 4D) and quantification analysis (FIG. 4E) of Ce6 cellular uptake levels in A375 cells treated with hybrid vesicles with different functional antibodies; FIG. 4F displays the fluorescence imaging and biodistribution of free Ce6, Ce6-loaded liposomes, and hybrid vesicles in A375 tumor-bearing nude mice in vivo at different timepoints after injection; FIG. 4G depicts the quantification results of Ce6 accumulation at tumor tissue area of different treatments at different time points after injection; FIG. 4H shows the fluorescence imaging of tumor tissues and major organs ex vivo 24 h after injection; and FIG. 4I depicts the quantification results of fluorescent intensity of Ce6 in the tumor tissues and major organs ex vivo 24 h after injection;

FIGS. 5A-5G depict that the photosensitive hybrid γδ-T exosomes induce synergistic cancer cell death, in which: FIG. 5A and FIG. 5B show the apoptosis assay of γδ-T exosomes to A375 cancer cells or HSF skin fibroblasts with different concentration; FIG. 5C and FIG. 5D display the apoptosis assay of hybrid γδ-T exosomes without light irradiation to A375 cancer cells or HSF skin fibroblasts with different concentrations; FIG. 5E depict the apoptosis assay of A375 cells after different treatments with or without light irradiation; FIG. 5F shows the percentage of PI-positive dead A375 cells after different treatments with or without light irradiation; and FIG. 5G depicts the colony formation assay and quantification results of A375 cells after different treatment;

FIGS. 6A-6G depict the ROS generation ability and cytolytic function of photosensitive hybrid γδ-T exosomes, in which: FIG. 6A and FIG. 6B depict the fluorescence spectra of hybrid γδ-T exosomes (FIG. 6A) or PBS buffer (FIG. 6B) with different light irradiation times using SOSG probe as the ¹O₂ sensor; FIG. 6C shows the fluorescence changes at 525 nm of hybrid γδ-T exosomes with SOSG probe or SOSG probe alone with different light irradiation times; FIG. 6D depicts the representative CLSM images of ROS generation inside A375 cells after different treatments with or without light irradiation; FIG. 6E shows the quantification results of ROS generation in A375 cells after different treatments with or without light irradiation; FIG. 6F and FIG. 6G show the analysis of cytolytic components within γδ-T exosomes or hybrid γδ-T exosomes;

FIGS. 7A-7K depict the DAMPs release and dendritic cell maturation triggered by photosensitive hybrid γδ-T exosomes, in which: FIG. 7A and FIG. 7B show the representative CLSM images and quantification results of CRT exposure on the A375 cells after different treatments with or without light irradiation; FIG. 7C and FIG. 7D depict the representative CLSM images and quantification results of HMGB1 release in the nucleus of A375 cells after different treatments with or without light irradiation; FIG. 7E depicts the representative CLSM images of colocalization between HSP60 and mitochondria inside A375 cells after different treatments with or without light irradiation; FIG. 7F shows the colocalization analysis between HSP60 and mitochondria inside A375 cells with different treatments; FIG. 7G depicts the relative ATP level inside A375 cells after different treatments with or without light irradiation; FIG. 7H and FIG. 7I show the flow cytometry analysis and quantification results of CD86 and HLA-DR on human dendritic cells; and FIG. 7J and FIG. 7K depict the level of MART-1-specific CD4⁺ T-cells (FIG. 7J) and CD8⁺ T-cells (FIG. 7K) in different treatment groups;

FIGS. 8A-8J depict the in vivo anti-melanoma growth effect of photosensitive hybrid γδ-T exosomes in A375 tumor-bearing nude mice, in which: FIG. 8A is a schematic illustration of tumor implantation and treatment schedule during antitumor study; FIGS. 8B-8E show the tumor volume growth curve of individual mice in PBS (FIG. 8B), exosomes (FIG. 8C), liposomes (FIG. 8D), and hybrid vesicles (FIG. 8E) groups; FIG. 8F depicts the tumor volume growth curve of the mice with different treatments; FIG. 8G shows the image of tumors isolated from A375 tumor-bearing nude mice; FIG. 8H shows the tumor weight of the mice with different treatments; FIG. 8I exhibits the hematoxylin and eosin (H&E) staining of the tumor tissue sections from A375 tumor-bearing nude mice with different treatments; and FIG. 8J shows the representative CLSM images of Ki67 and TUNEL in the tumor tissue sections from A375 tumor-bearing nude mice after different treatments;

FIG. 9 depicts the hemolysis assay of hybrid Vδ2 T exosomes with different concentrations;

FIG. 10 depicts the H&E staining of the main organ (heart, kidney, liver, lung, and spleen) sections from A375 tumor-bearing nude mice with different treatments;

FIG. 11 depicts the body weight change of A375 tumor-bearing nude mice with different treatments; and

FIGS. 12A-12B depict the serum levels of ALT (FIG. 12A) and AST (FIG. 12B) of A375 tumor-bearing nude mice with different treatments.

### DETAILED DESCRIPTION

In the following description, photo-sensitive hybrid vesicles and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

As used herein, the term “γδ-T exosomes” refer to extracellular vesicles secreted by human or animal γδ-T lymphocytes. These vesicles typically range from about 20 nm to 200 nm in diameter, originate from endosomal compartments, and naturally contain lipids, proteins, nucleic acids, and surface markers derived from the parent γδ-T cells (e.g., CD63, γδ-TCR, MHC molecules, and co-stimulatory molecules). γδ-T exosomes retain functional properties of γδ-T cells, including the ability to induce tumor cell apoptosis and modulate immune responses.

As used herein, the term “photosensitizer-loaded liposome” refers to a liposomal nanoparticle in which a photosensitizing agent (such as chlorin e6, porphyrins, phthalocyanines, or other light-activated molecules) is encapsulated within the aqueous core and/or intercalated into the lipid bilayer of the liposome. The liposome typically comprises one or more biocompatible phospholipids, cholesterol, and optional surface-modifying agents such as polyethylene glycol (PEG) conjugates to enhance stability, circulation time, and targeting. Upon exposure to light of an appropriate wavelength, the encapsulated photosensitizer becomes activated and produces ROS to induce localized cytotoxic effects. These liposomes are designed to combine the therapeutic effect of photodynamic therapy with the favorable pharmacokinetics, protection, and delivery properties of liposomal carriers.

As used herein, the term “photosensitive hybrid vesicles” refer to hybrid vesicular structures obtained by fusing γδ-T exosomes with photosensitizer-loaded liposomes (e.g., Ce6-loaded liposomes). These hybrid vesicles combine the biological targeting and immunomodulatory characteristics of γδ-T exosomes with the light-activated therapeutic properties of the photosensitizer in the liposomal component. The resulting vesicles typically exhibit a single lipid bilayer with retained exosomal surface markers and encapsulated cytolytic or photosensitizing agents, enabling targeted delivery and light-triggered cytotoxicity.

In accordance with a first aspect of the present invention, a photosensitive hybrid vesicle is provided. The photosensitive hybrid vesicle is designed to combine the therapeutic functionalities of γδ-T exosomes with the light-activated properties of photosensitizer-loaded liposomes. In particular, the hybrid vesicle is constructed by fusing a γδ-T exosome with a photosensitizer-loaded liposome via a membrane fusion process, thereby creating a single delivery vehicle that integrates both cellular and synthetic components. The resulting hybrid vesicle comprises a lipid bilayer structure, closely mimicking biological membranes, which enhances its stability and biocompatibility under physiological conditions.

A key feature of the hybrid vesicle is its ability to retain functional surface proteins and cytolytic molecules derived from the γδ-T exosome while simultaneously incorporating the photosensitizer from the photosensitizer-loaded liposome. This unique combination enables the vesicle to generate ROS upon exposure to light, thus allowing for PDT while preserving the inherent cytolytic activity of γδ-T exosomes. More specifically, the cytolytic molecules retained within the hybrid vesicle include granzyme A, granzyme B, perforin, and granulysin, which are known to mediate direct tumor cell killing. In addition, the functional surface proteins carried over from the γδ-T exosome, such as PD-1 and CCR5, facilitate active targeting and interaction with tumor cells, thereby enhancing cellular uptake and therapeutic specificity.

The hybrid vesicle may further includes one or more additional therapeutic agents to achieve combination therapy. Suitable therapeutic agents include, but are not limited to, chemotherapeutic agents, monoclonal antibodies, immune checkpoint inhibitors, immunomodulatory cytokines, small interfering RNA (siRNA) or microRNA (miRNA), gene-editing cargos such as CRISPR/Cas components, radiotherapeutic agents, targeted small-molecule inhibitors, and oncolytic viruses. This versatility allows the hybrid vesicle to function as a multimodal platform capable of delivering diverse therapeutic payloads.

The photosensitizer incorporated into the liposomal component may be Ce6, a porphyrin, phthalocyanine, a bacteriochlorin derivative, or any combination thereof. The hybrid vesicle is formulated to deliver hydrophobic drugs, hydrophilic drugs, or both simultaneously, providing flexibility for loading a wide range of therapeutic agents. The membrane fusion between the γδ-T exosome and the photosensitizer-loaded liposome may be achieved by various techniques including freeze-thaw processes, polyethylene glycol-mediated fusion, natural incubation, or extrusion-based fusion, depending on the desired fusion efficiency and preservation of functional biomolecules.

The resulting hybrid vesicle typically exhibits an average particle size ranging from about 50 nm to about 300 nm and maintains structural stability under physiological conditions for at least 48 hours. This stability ensures effective circulation and bioavailability during systemic administration. In some embodiments, the vesicle may further comprise targeting ligands or peptides to enhance tissue or tumor specificity. Such targeting moieties may be selected from antibodies, antibody fragments, peptides, aptamers, or small molecules that specifically bind to tumor-associated antigens or receptors, thereby enabling precision delivery to cancerous tissues while minimizing off-target effects.

Through the construction, the photosensitive hybrid vesicle combines the cell-derived targeting and cytolytic functions of γδ-T exosomes with the light-activated ROS-generating properties of photosensitizer-loaded liposomes in a single nanoscale platform. This synergistic design enables enhanced tumor accumulation, selective cytotoxicity upon light activation, and the possibility of incorporating additional therapeutic agents for improved treatment outcomes.

In accordance with a second aspect of the present invention, a pharmaceutical composition including the aforementioned photosensitive hybrid vesicle in combination with a pharmaceutically acceptable addition is provided. By formulating the hybrid vesicle into a pharmaceutical composition, the invention enables its administration in a manner suitable for therapeutic use in humans or animals.

The pharmaceutically acceptable addition in the composition may comprise one or more of an excipient, a stability additive, a carrier, a diluent, or a solubilizer. These additional components serve to enhance the stability, solubility, bioavailability, or case of administration of the hybrid vesicle without altering its fundamental biological or photosensitive properties. For example, the composition may include pharmaceutically acceptable carriers to facilitate parenteral administration, stabilizers to maintain the structural integrity of the vesicle during storage, and diluents or solubilizers to adjust concentration or improve dispersion. This formulation strategy allows the photosensitive hybrid vesicle to be provided as a ready-to-use therapeutic product for treating cancer or other conditions where a combination of γδ-T exosome activity and photosensitizer-mediated ROS generation is advantageous.

In accordance with a third aspect of the present invention, a method for treating cancer in a subject using the pharmaceutical composition including the photosensitive hybrid vesicle described above. The method involves administering to the subject a therapeutically effective amount of the pharmaceutical composition. After administration, a tumor tissue area of the subject is exposed to light of a wavelength sufficient to activate the photosensitive hybrid vesicle, thereby initiating the generation of ROS and the cytolytic activity of the vesicle within the tumor tissue.

The treatment method is suitable for a wide range of cancers, including but not limited to melanoma, retinoblastoma, colon cancer, breast cancer, gastric cancer, pancreatic cancer, lung cancer, and other solid tumors. By virtue of the functional surface proteins and cytolytic molecules retained from the γδ-T exosome and the incorporated photosensitizer from the liposome, the hybrid vesicle exerts both a targeted delivery effect and a photoresponsive therapeutic effect when activated by light.

The wavelength of light applied to the tumor tissue may be between 630 nm and 680 nm, which effectively excites photosensitizers such as Ce6, porphyrins, or phthalocyanines loaded into the vesicle to generate ROS and induce apoptosis or necrosis of tumor cells. The light exposure can be carried out externally or internally depending on the location of the tumor tissue and clinical requirements.

The pharmaceutical composition can be administered via various routes depending on the indication and tumor accessibility. In certain embodiments, the composition is delivered intravenously to achieve systemic distribution to tumor sites. In other embodiments, it may be administered intratumorally to maximize local concentration within the tumor, or topically when the tumor is superficial, such as in cutaneous lesions. This flexibility in administration allows the method to be adapted for different cancer types and clinical settings while maintaining the ability of the hybrid vesicle to exert its synergistic cytolytic and photosensitive activity once activated by light.

In accordance with a fourth aspect of the present invention, a method of manufacturing the aforementioned photosensitive hybrid vesicle is introduced. In the method, a photosensitizer-loaded liposome is first prepared, for example by established lipid film hydration and extrusion techniques. In parallel, a γδ-T exosome is isolated from γδ-T cells, such as PAM-expanded human γδ-T lymphocytes, by centrifugation or ultracentrifugation of conditioned culture medium and subsequent purification steps. The photosensitizer-loaded liposome and the γδ-T exosome are then fused together by a membrane fusion technique to form a hybrid vesicle that integrates structural and functional components from both parental systems. The fusion may be carried out using physical or chemical procedures, including but not limited to freeze-thaw cycling, polyethylene glycol-mediated fusion, natural incubation, or extrusion-based fusion methods, with conditions selected to maximize fusion efficiency while preserving exosomal proteins and liposomal drug cargo.

The process yields a photosensitive hybrid vesicle that retains functional surface proteins and cytolytic molecules from the γδ-T exosome and incorporates the photosensitizer from the photosensitizer-loaded liposome for generating reactive oxygen species upon light irradiation. In particular, the resulting hybrid vesicles preserve the characteristic cytolytic molecules such as granzyme A, granzyme B, perforin and granulysin and maintain key functional surface proteins including PD-1 and CCR5. These molecules and proteins are inherited from the γδ-T exosomes and are crucial for mediating the vesicle's tumor-targeting capability and cytotoxic activity. By combining the exosomal proteins and cytolytic molecules with the photosensitizer component of the liposome in a single hybrid vesicle, the manufacturing method produces a nanoparticle that is capable of both targeted delivery and potent photoinduced therapeutic action.

### EXAMPLES

### Example 1. Fabrication of Photosensitive Hybrid Vesicles Via Membrane Fusion of Ce6-Loaded Liposomes and γδ-T Exosomes

Photosensitive hybrid vesicles are fabricated by fusing Ce6-loaded liposomes with γδ-T exosomes. The γδ-T exosomes are harvested by ultracentrifugation from the conditioned medium of PAM-expanded human γδ-T cells following standard protocols, while the Ce6-loaded liposomes are prepared using a thin-film hydration method.

Briefly, Ce6-loaded liposomes composed of HSPC (54 mol %), cholesterol (30 mol %), DSPE-PEG2K (5 mol %), and Ce6 (11 mol %) are prepared by dissolving the lipid mixture and Ce6 in a methanol/chloroform (3:1) solution. The solvent is evaporated to form a thin lipid film on the bottom of a round-bottom flask, which is then hydrated with PBS buffer at 56° C. for 30 min to form liposomes. The resulting liposomes are sequentially extruded through 400 nm, 200 nm, and 100 nm polycarbonate filters to reduce particle size. Unencapsulated Ce6 is removed by ultracentrifugation at 60,000 g for 1 h. To prepare Nile Red-loaded or FRET liposomes, Nile Red or DSPE-Rhodamine B is incorporated during the initial lipid/solvent step, followed by evaporation, hydration, and extrusion as described above.

For γδ-T cell expansion and exosome isolation, human PBMCs are isolated from buffy coats of healthy donors (Hong Kong Red Cross) using Ficoll-Hypaque gradient centrifugation. PBMCs are expanded with PAM and recombinant human IL-2 for 14 days. The expanded γδ-T cells are washed with PBS and cultured in exosome-free 10% FBS-RPMI 1640 medium supplemented with IL-2 for 48 h. The conditioned medium is centrifuged at 2,000 g for 20 min at 4° C. to remove cells, then at 10,000 g for 30 min to remove debris. The supernatant is passed through a 0.22 μm syringe filter and ultracentrifuged at 100,000 g for 70 min at 4° C. The resulting exosome pellet is resuspended in cold PBS (pH 7.4) and stored at −80° C.

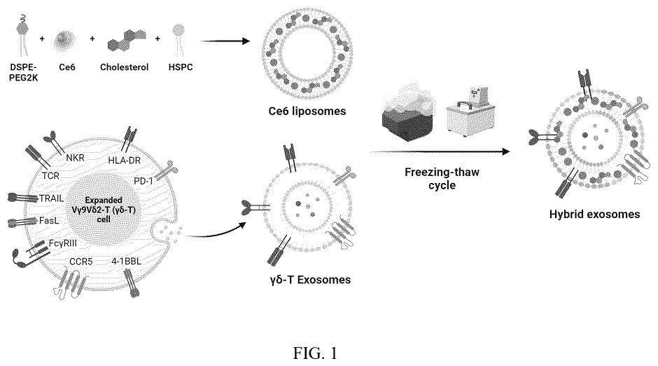

Previous studies have demonstrated several chemical and physical methods for fusing liposomes with natural exosomes, including freeze-thaw fusion, incubation, polyethylene glycol-mediated fusion, and membrane extrusion. To optimize fusion efficiency, drug loading, and membrane integrity, the freeze-thaw method is selected for fusing γδ-T exosomes with Ce6-loaded liposomes (FIG. 1). In brief, the two vesicle types are mixed at a 1:1 (w/w) ratio and subjected to multiple freeze-thaw cycles. The resulting hybrid vesicles are purified by ultrafiltration to remove debris.

During freezing, ice crystal formation transiently disrupts the lipid bilayers of γδ-T exosomes and Ce6-liposomes, and subsequent thawing promotes fusion of these disrupted bilayers. This process produces hybrid vesicles that retain both Ce6 and exosomal cytolytic molecules internally, as well as functional membrane markers externally.

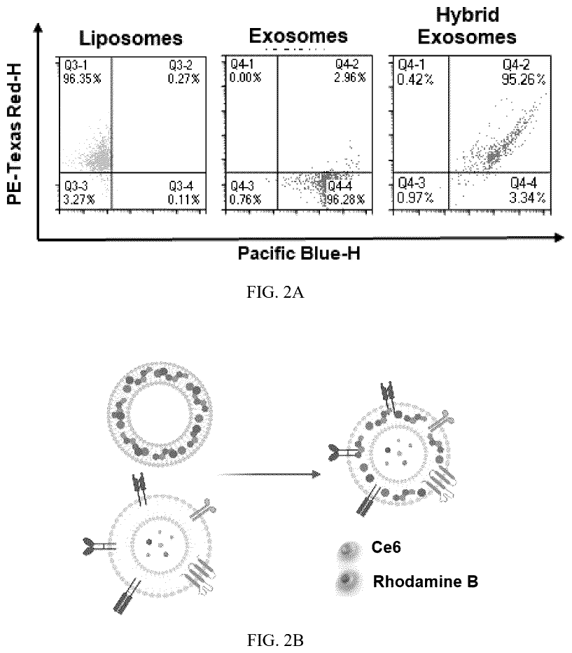

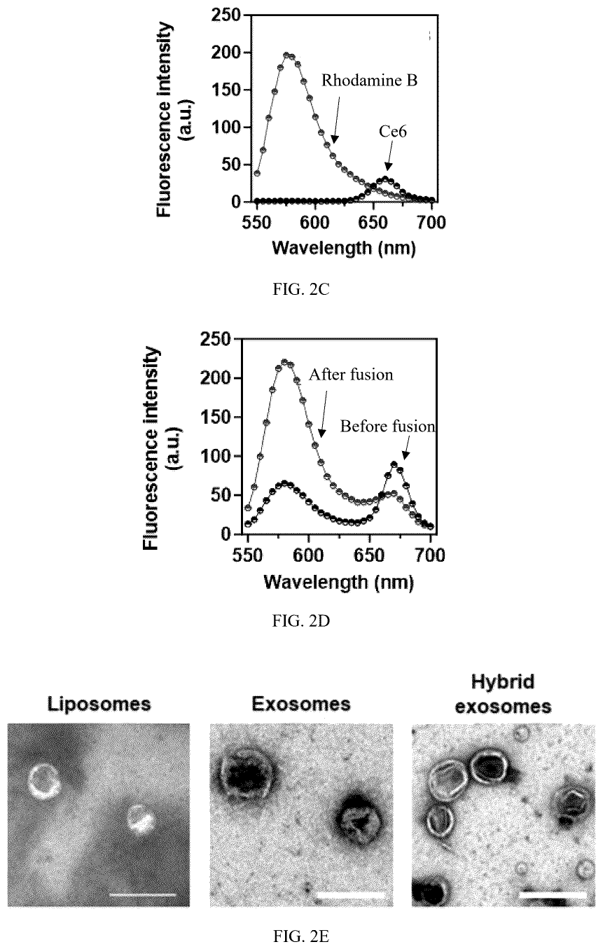

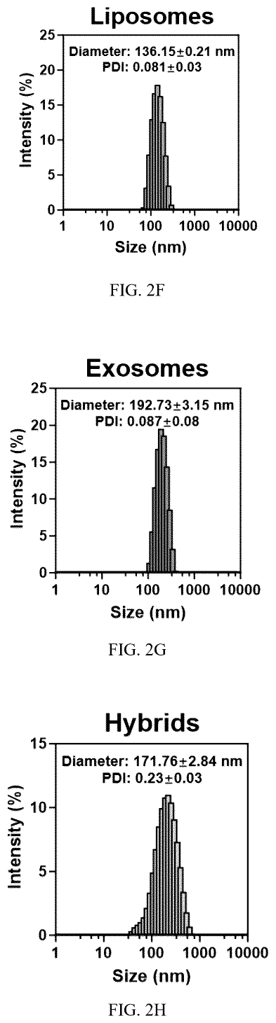

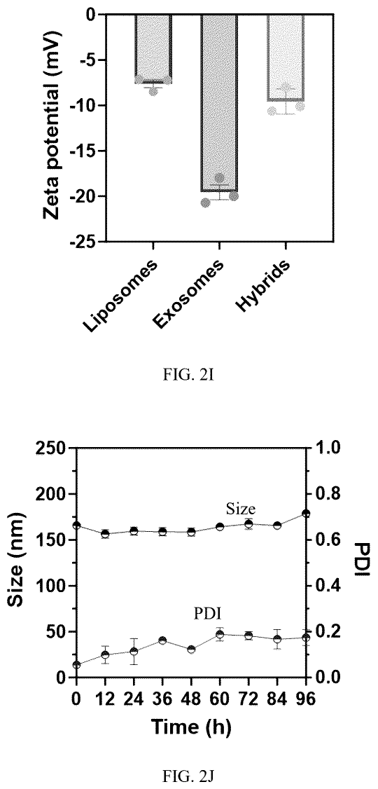

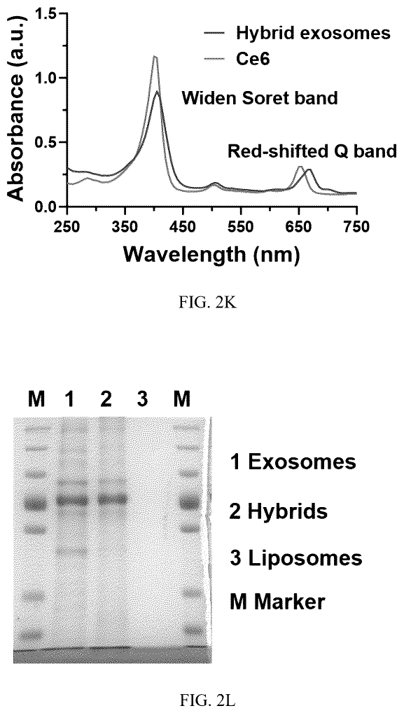

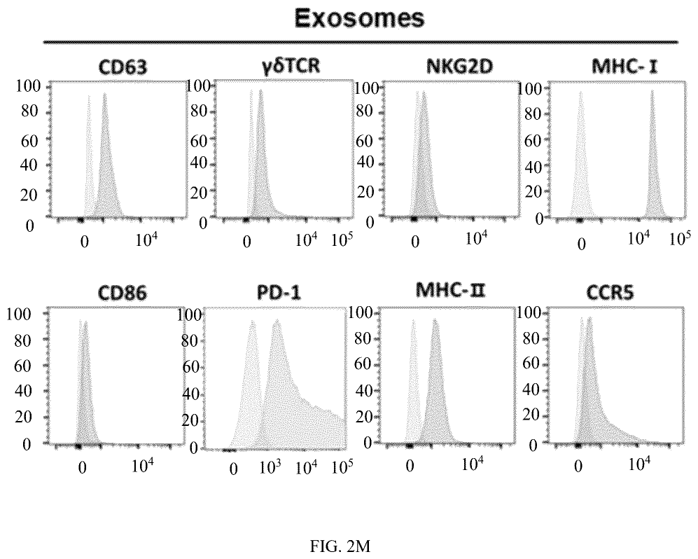

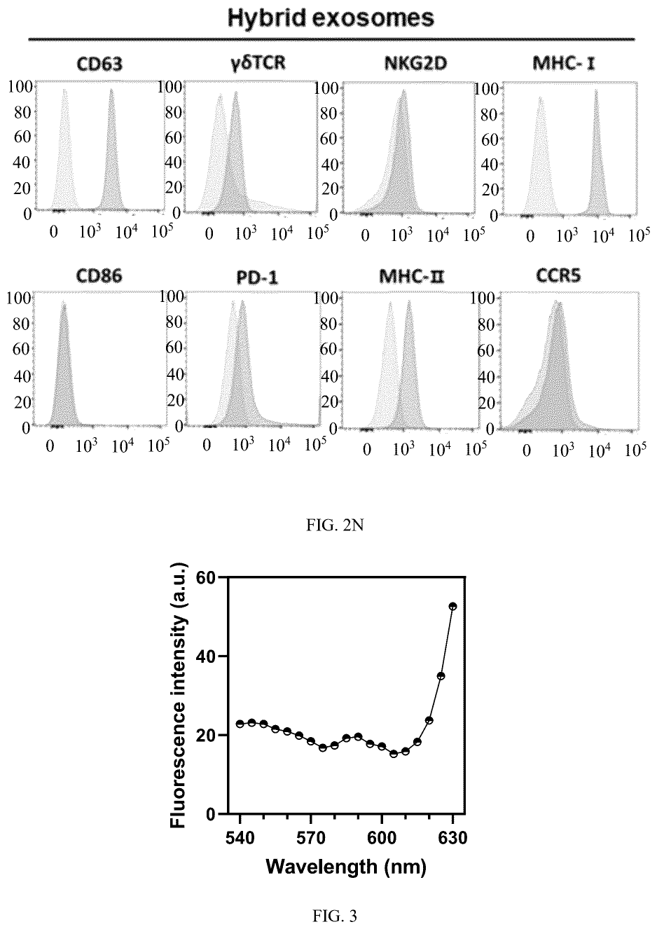

Fusion efficiency is evaluated by small particle flow cytometry and Förster Resonance Energy Transfer (FRET). Liposomes are preloaded with Nile Red, and γδ-T exosomes are stained with CFSE-Pacific Blue. After freeze-thaw cycles, over 95% of nanoparticles are double-positive for PE-Texas Red and Pacific Blue, indicating >95% fusion efficiency (FIG. 2A). To further confirm membrane fusion, FRET liposomes containing DSPE-Rhodamine B and Ce6 are mixed with γδ-T exosomes. Post-fusion, FRET activity decreases markedly (FIG. 2D), consistent with increased donor-acceptor separation and confirming insertion of γδ-T exosomal contents into liposomal bilayers (FIG. 2B-C, FIG. 3).

Transmission electron microscopy (TEM) reveals intact cup-shaped hybrid vesicles showing features of both Ce6-liposomes and γδ-T exosomes (FIG. 2E). Dynamic light scattering (DLS) shows particle sizes of ˜136 nm (Ce6-liposomes) and ˜192 nm (γδ-T exosomes), while the hybrid vesicles average 171.76 nm with a polydispersity index (PDI) of 0.23 (FIG. 2F-H). The zeta potential of hybrid vesicles (−9.57 mV) lay between those of the parent vesicles (FIG. 2I). The hybrid vesicles remain stable in physiological conditions for at least 48 h (FIG. 2J). Ce6 encapsulation efficiency and loading capacity are ˜38% and 5%, respectively, as determined by HPLC. UV-Vis spectra exhibit a broadened Soret band at 400 nm and a red-shifted Q band at 650 nm compared with free Ce6, indicating strong Ce6-lipid interactions and successful encapsulation (FIG. 2K).

$$Encapsulation efficiency ( \% ) = \frac{W eight of loaded Ce 6}{W eight of fed Ce 6} \times 100 \%$$

$$Loading capacity ( \% ) = \frac{W eight of loaded Ce 6}{W eight of hybrid vesicles} \times 100 \%$$

Protein composition is examined by SDS-PAGE. Equal protein amounts from exosomes and hybrid vesicles are run on 10% SDS-PAGE gels, stained with Coomassie Blue for 1 h, washed overnight, and imaged using an Amersham Imager 680. The protein profiles of γδ-T exosomes are largely retained in hybrid vesicles (FIG. 2L).

In addition, the preservation of specific functional proteins on the surface of hybrid vesicles is further identified. Briefly, the surface marker analysis of γδ-T exosomes and hybrid vesicles is conducted by incubating exosomes or hybrid vesicles with 4-μm aldehyde/sulfate latex beads overnight and blocking remaining binding sites. Then, the blocked beads are stained with fluorophore-conjugated flow antibodies against CD63, γδTCR, NKG2D, MHC-I, CD86, PD-1, MHC-II, and CCR5 or corresponding matched isotype controls. The surface expression of functional proteins is then analyzed under the NovoCyte Quanteon (Agilent). As shown in FIG. 2M and FIG. 2N, both hybrid vesicles and γδ-T exosomes display CD63 (exosome marker), γδ-TCR, major histocompatibility complex-1 (MHC-I), CD86, programmed cell death protein 1 (PD-1), MHC-II, C-C chemokine receptor type 5 (CCR5) (functional markers) on the surface, demonstrating these proteins are preserved during membrane fusion and hybrid vesicles might share similar therapeutic functions with γδ-T exosomes.

Collectively, these results demonstrate that the hybrid vesicles successfully integrate the properties of γδ-T exosomes and Ce6-loaded liposomes, potentially conferring active targeting and photosensitive apoptotic activity derived from both components.

### Example 2. Photosensitive Hybrid Vesicles Promote Drug Accumulation within Human Melanoma Tissues

Based on previous studies demonstrating that γδ-T exosomes exhibit intrinsic tumor-targeting properties, it is hypothesized that the hybrid vesicles would similarly possess active targeting capability toward melanoma cells. To evaluate this, the Ce6 uptake efficiency of free Ce6, Ce6-loaded liposomes, and hybrid vesicles is compared in A375 melanoma cells. Equal amounts of Ce6 are administered to A375 cells, and after four hours, Ce6 uptake is quantified using a NovoCyte Quanteon flow cytometer. For confocal laser scanning microscopy (CLSM) analysis, A375 cells are washed with PBS three times and stained with DAPI prior to imaging. To investigate the mechanism underlying the active targeting of the hybrid vesicles, functional blocking antibodies against PD-1 or CCR5, or corresponding isotype controls, are introduced into the coculture system and Ce6 uptake is measured after four hours using the NovoCyte Quanteon.

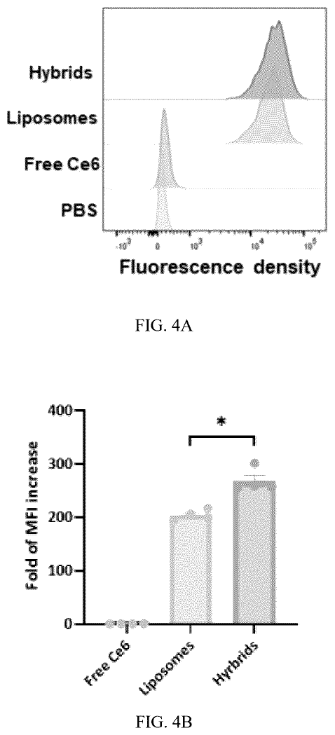

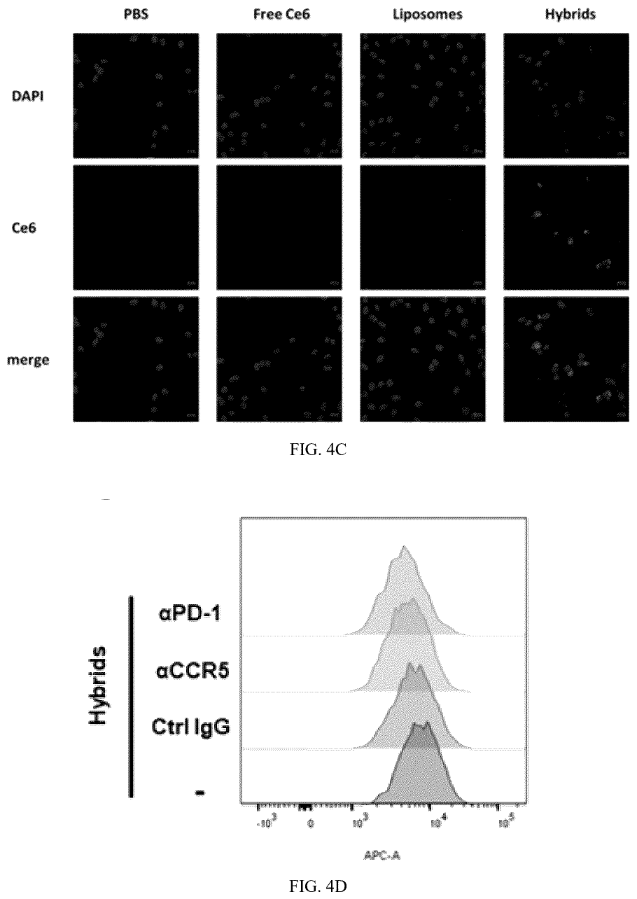

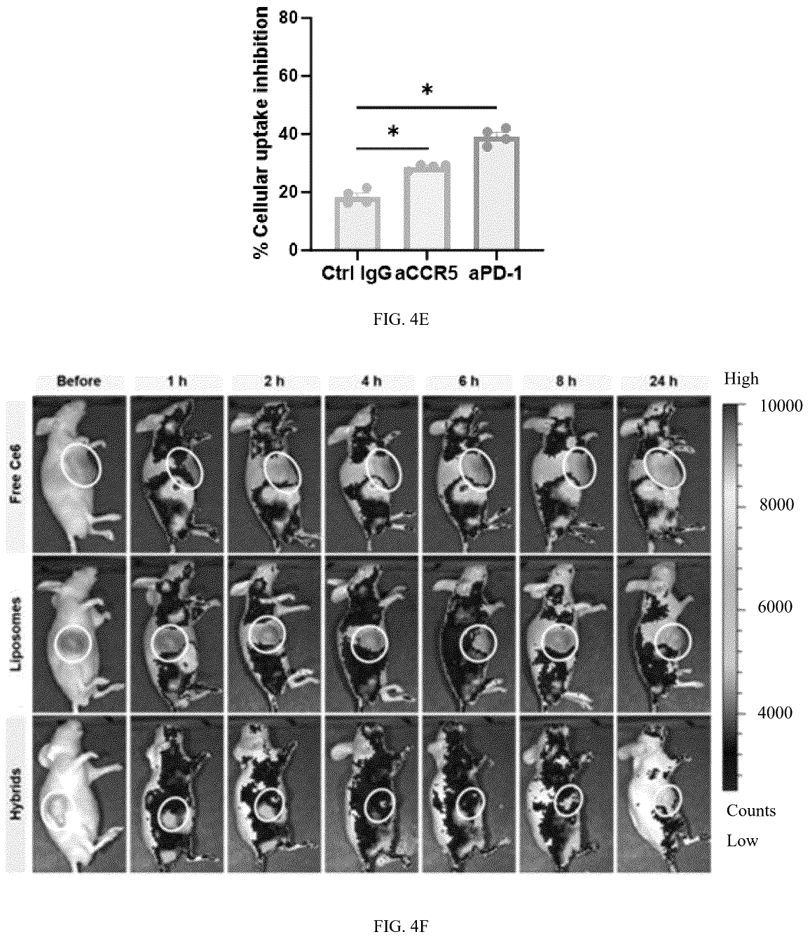

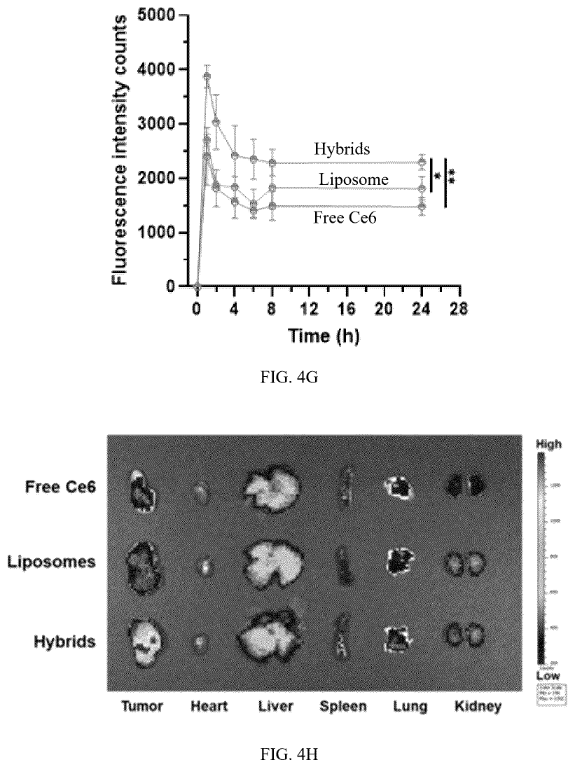

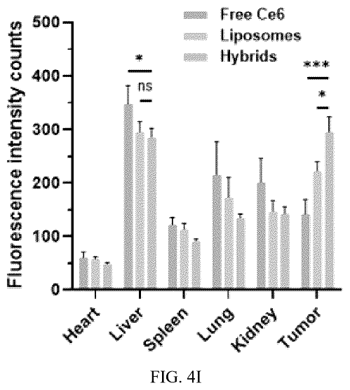

Flow cytometric analysis revealed that hybrid vesicles markedly enhance intracellular Ce6 accumulation compared with Ce6-loaded liposomes, indicating their superior drug delivery capability (FIG. 4A and FIG. 4B). CLSM imaging confirms these results, showing stronger intracellular fluorescence and more abundant red puncta in hybrid vesicle-treated cells than in liposome-treated cells (FIG. 4C). Blocking experiments demonstrate that this increased uptake is mediated, at least in part, by CCR5 and PD-1 surface proteins: neutralizing either CCR5 or PD-1 with monoclonal antibodies significantly reduced hybrid vesicle uptake compared to the isotype control (FIG. 4D and FIG. 4E). These results suggest that functional surface markers on the hybrid vesicles are responsible for their active targeting effect and enhanced drug delivery to melanoma cells.

To further assess this targeting effect in vivo, an A375 xenograft tumor model is established in nude mice. Human A375 melanoma cells are inoculated subcutaneously, and once tumors reach approximately 800 mm³, mice receive intravenous injections of free Ce6, Ce6-loaded liposomes, or hybrid vesicles containing equivalent amounts of Ce6. Whole-body fluorescence images are captured at 0, 1, 2, 4, 6, 8, and 24 hours post-injection using the PE IVIS Spectrum system (n=3 per group). After 24 hours, mice are sacrificed, and the fluorescence intensity of Ce6 in tumors and major organs (heart, liver, spleen, lung, and kidney) is analyzed with IVIS.

In vivo imaging revealed that hybrid vesicles achieve substantially greater tumor accumulation than free Ce6 or Ce6-loaded liposomes at all time points measured (FIG. 4F and FIG. 4G). Ex vivo imaging of tumors and organs at 24 hours confirms these findings: hybrid vesicles primarily accumulate in both tumor tissue and the liver (FIG. 4H). Quantitative analysis shows that hybrid vesicles achieve 2.12-fold higher Ce6 accumulation in tumor tissue compared to free Ce6 and 1.35-fold higher compared to liposomes (FIG. 4I). Taken together, these in vitro and in vivo results demonstrate that hybrid vesicles possess a pronounced active targeting effect toward melanoma tissues, likely mediated by surface markers such as CCR5 and PD-1, thereby improving drug delivery to tumor sites.

### Example 3. Photosensitive Hybrid Vesicles Induce Synergistic Cytotoxicity Effect Against Human Melanoma Cells

To evaluate the antitumor potential of the hybrid vesicles, the cytotoxic effects of hybrid vesicles on human melanoma cells and normal cells are first examined. Human melanoma A375 cells and HSF are treated with γδ-T exosomes, Ce6-loaded liposomes, or hybrid vesicles, with or without light irradiation (Xe Lamp, 650 nm, 6.4 mW/cm², 12 min). After 24 hours, cells are harvested and stained with FITC-Annexin V/PI in binding buffer at room temperature for 15 minutes and analyzed directly using a NovoCyte Quanteon flow cytometer.

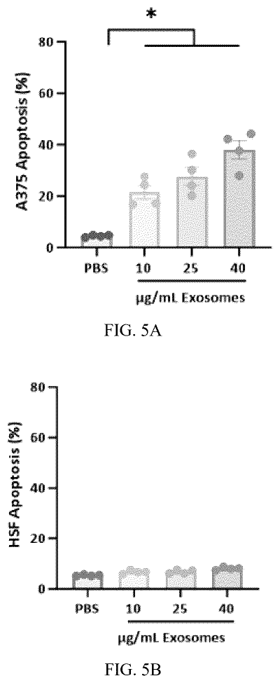

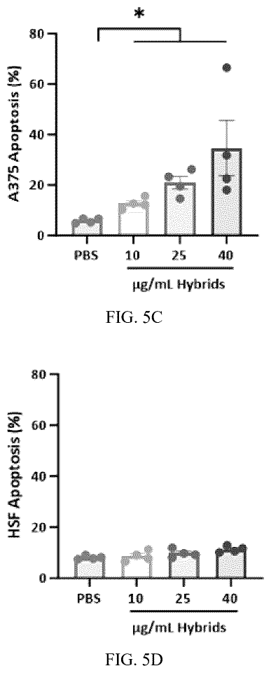

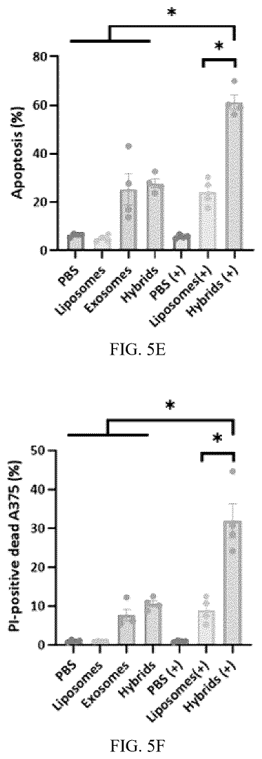

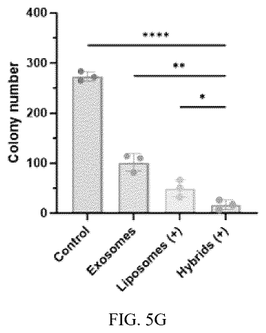

Flow cytometry results show that γδ-T exosomes induce dose-dependent apoptosis in A375 tumor cells (FIG. 5A). Importantly, minimal apoptosis is observed in normal HSF cells across all tested concentrations, indicating the exosomes' selective cytotoxicity for melanoma cells (FIG. 5B). Likewise, hybrid vesicles without light irradiation exhibit dose-dependent cytotoxicity toward tumor cells but maintained high biocompatibility toward normal cells (FIG. 5C and FIG. 5D). To assess the combined antitumor effect of γδ-T exosomes and photodynamic therapy (PDT), A375 cells are treated with liposomes, γδ-T exosomes, or hybrid vesicles with or without light exposure. Under dark conditions, exosomes and hybrid vesicles show comparable cytotoxicity against A375 cells. However, upon red light irradiation (Xe Lamp, 650 nm, 6.4 mW/cm², 12 min), Ce6-loaded liposomes produce increased cell apoptosis and death comparable to that induced by γδ-T exosomes without light exposure. Strikingly, hybrid vesicles cause more than a twofold increase in melanoma cell apoptosis and death compared to γδ-T exosomes or liposomes under the same conditions, demonstrating a potent synergistic antitumor effect (FIG. 5E and FIG. 5F).

The synergistic effect is further validated by colony formation assays. A375 cells are seeded in 6-well plates (800 cells per well) and treated with γδ-T exosomes, hybrid vesicles, or Ce6-loaded liposomes, with or without light irradiation (Xe Lamp, 650 nm, 6.4 mW/cm², 12 min). After 7 days, cells are fixed with 4% PFA and stained with 0.1% crystal violet. Following PBS washes, colony formation is imaged.

As shown in FIG. 5G, hybrid vesicles combined with light irradiation exhibit a markedly stronger anti-clonogenic effect compared to all other groups. Collectively, these findings demonstrate that hybrid vesicles achieve a photoresponsive synergistic cytotoxic effect against melanoma cells, exceeding the efficacy of either component alone.

### Example 4. ROS Generation and Cytolytic Function of Photosensitive Hybrid Vesicles

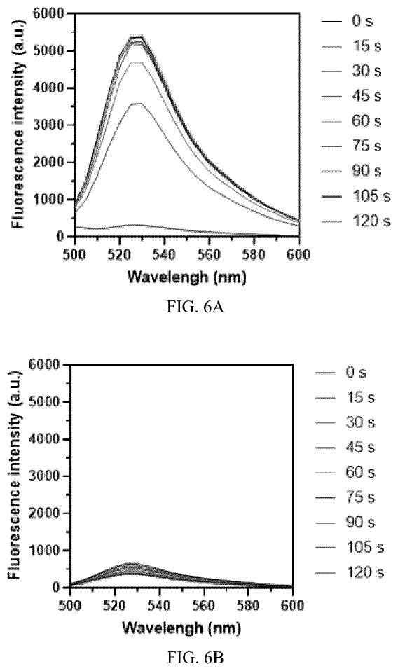

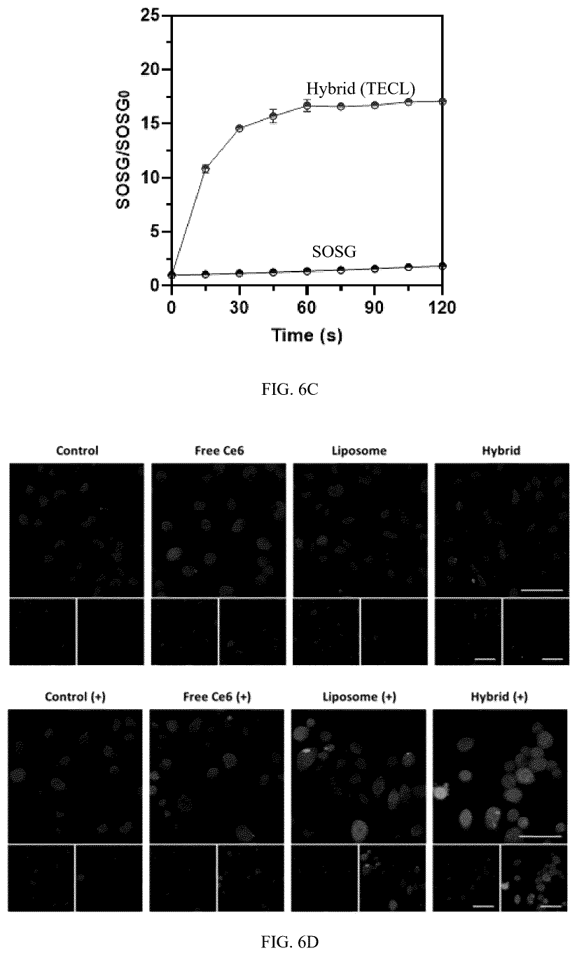

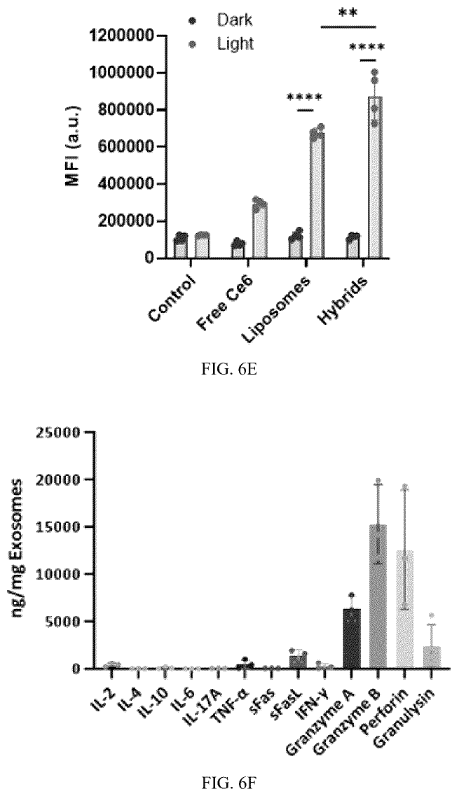

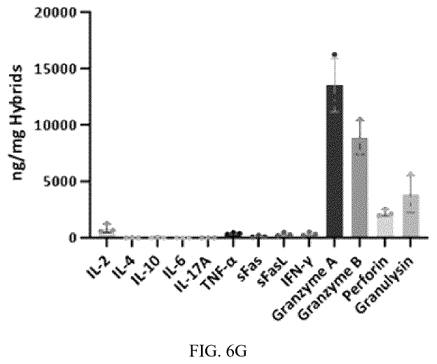

To determine whether the photoresponsive cancer cell death induced by hybrid vesicles arises from photodynamic therapy (PDT)-mediated reactive oxygen species (ROS) generation, the production of singlet oxygen (¹O₂) is examined using the SOSG probe. Hybrid vesicles are mixed with SOSG probe (5 μM) in PBS, and the fluorescence spectrum (excitation at 488 nm, emission 500-600 nm) and the 525 nm signal are measured with a multi-mode microplate reader at different time points under light irradiation (LED, 650 nm, 30 mW/cm², 0-120 s). Upon red light exposure, hybrid vesicles produce substantial amounts of ¹O₂ in a time-dependent manner (FIGS. 6A-6C).

Intracellular ROS levels are evaluated in A375 melanoma cells using the 2′,7′-dichlorodihydrofluorescein diacetate (DCFH-DA) probe under CLSM. Human melanoma cells A375 are seeded in confocal dishes and treated with γδ-T exosomes, hybrid vesicles, or Ce6-loaded liposomes for 4 hours. Cells are then exposed to or kept in the dark without light irradiation (Xe Lamp, 650 nm, 6.4 mW/cm², 12 min). After washing with PBS, A375 cells are stained with DCFH-DA and Hoechst 33342 and imaged by CLSM. For flow cytometry, A375 cells after treatment are stained with DCFH-DA and analyzed using a NovoCyte Quanteon flow cytometer.

Following light irradiation, hybrid vesicles generate markedly higher levels of ROS in both the cytoplasm and nucleus compared with dark conditions (FIG. 6D). Flow cytometric analysis corroborates these results, showing significantly increased ROS production in irradiated hybrid vesicle-treated cells relative to all other groups (FIG. 6E), suggesting ROS generation is a key mechanism underlying the photoresponsive cancer cell apoptosis induced by hybrid vesicles.

Beyond ROS generation, the antitumor activity of hybrid vesicles may also derive from cytolytic molecules inherited from γδ-T exosomes. Therefore, LEGENDplex analysis is performed to quantify cytolytic molecules. γδ-T exosomes and hybrid vesicles are diluted in the assay buffer of LEGENDplex Human CD8/NK Panel V02 (Biolegend), processed per the manufacturer's instructions, and analyzed online via the Biolegend platform.

As shown in FIG. 6F, γδ-T exosomes contain high levels of cytolytic molecules such as granzyme A, granzyme B, perforin, and granulysin but minimal levels of other cytokines, including IL-2, IL-4, IL-10, IL-6, IL-17A, TNF-α, soluble FasL, and IFN-γ. Importantly, most of these cytolytic molecules remain present in the hybrid vesicles after membrane fusion and nanoformulation (FIG. 6G), explaining their intrinsic cytotoxicity toward A375 cells even without light irradiation. Collectively, these results demonstrate that the enhanced photosensitive antitumor effect of hybrid vesicles results from a synergistic mechanism combining ROS generation and cytolytic molecules derived from parental activated γδ-T cells.

### Example 5. Photoresponsive Hybrid Vesicles Trigger DAMP Release and Induce Tumor Antigen-Specific αβ-T Cell Response

It is well established that ROS generated by photodynamic therapy (PDT) within cancer cells can induce endoplasmic reticulum (ER) stress-related immunogenic cell death (ICD). ICD is characterized by the upregulation and release of damage-associated molecular patterns (DAMPs), which promote dendritic cell maturation, cytotoxic αβ-T cell activation, and systemic antitumor immunity. Because hybrid vesicles have demonstrated photosensitive ROS production and tumor cell apoptosis, it is hypothesized that they may also trigger ICD in A375 melanoma cells under light irradiation. Two hallmark DAMP markers are evaluated: calreticulin (CRT), an ER-resident chaperone, and high mobility group box 1 (HMGB1), a nuclear protein involved in nucleosome maintenance.

Human melanoma A375 cells are seeded in confocal dishes and treated with γδ-T exosomes, hybrid vesicles, or Ce6-loaded liposomes, with or without light irradiation (Xe Lamp, 650 nm, 6.4 mW/cm², 12 min). After 24 hours, cells are fixed with 4% PFA for 15 minutes, washed three times with PBS, permeabilized with 0.2% Triton X-100, and blocked with 3% BSA in PBS for 1 hour at room temperature. Cells are incubated overnight at 4° C. with anti-CRT or anti-HMGB1 primary antibodies, washed, then stained with FITC-conjugated secondary antibodies for 1 hour at room temperature. After a final PBS wash, cells are counterstained with DAPI and mounted with anti-fade fluorescence medium (ab104135, Abcam). Quantification is performed using ImageJ software.

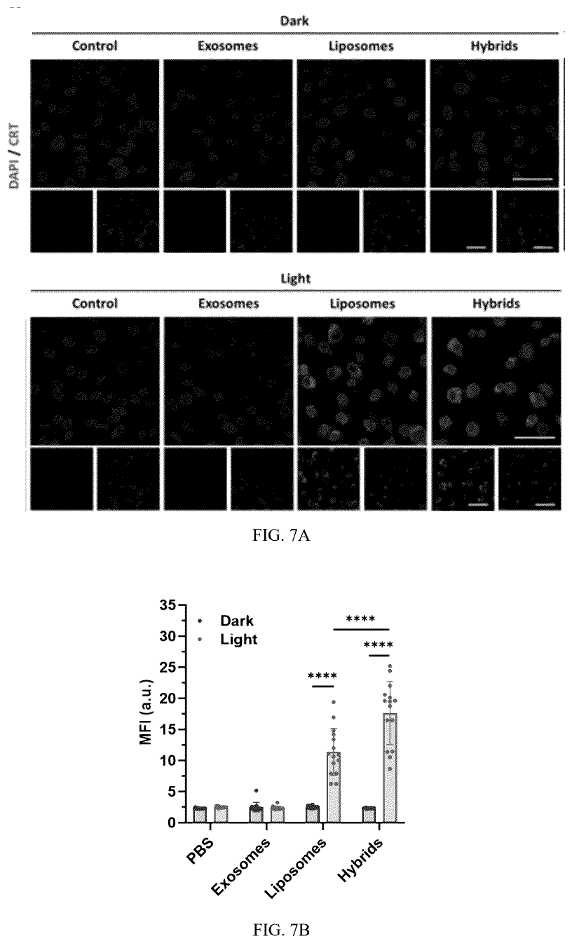

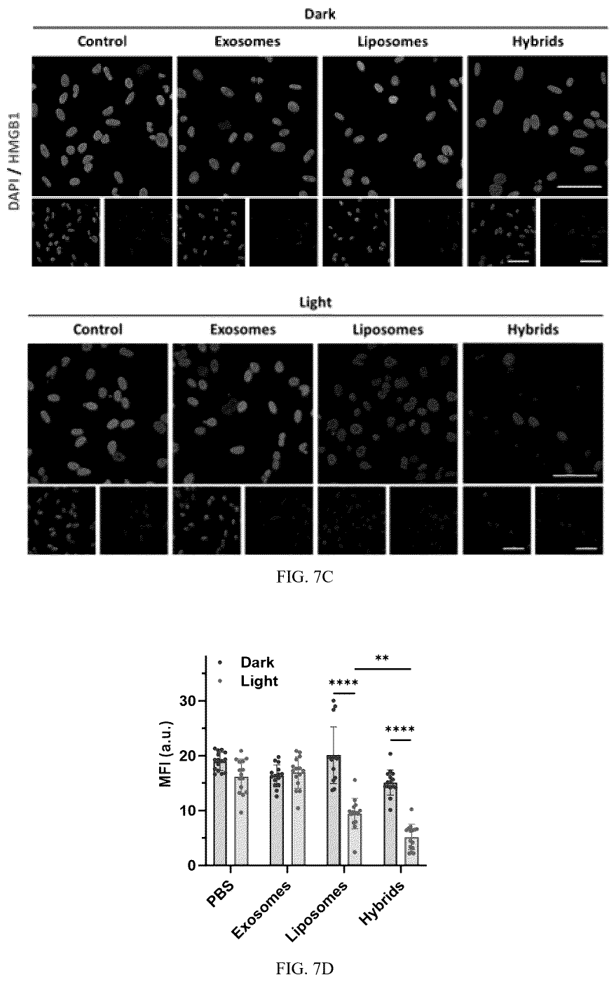

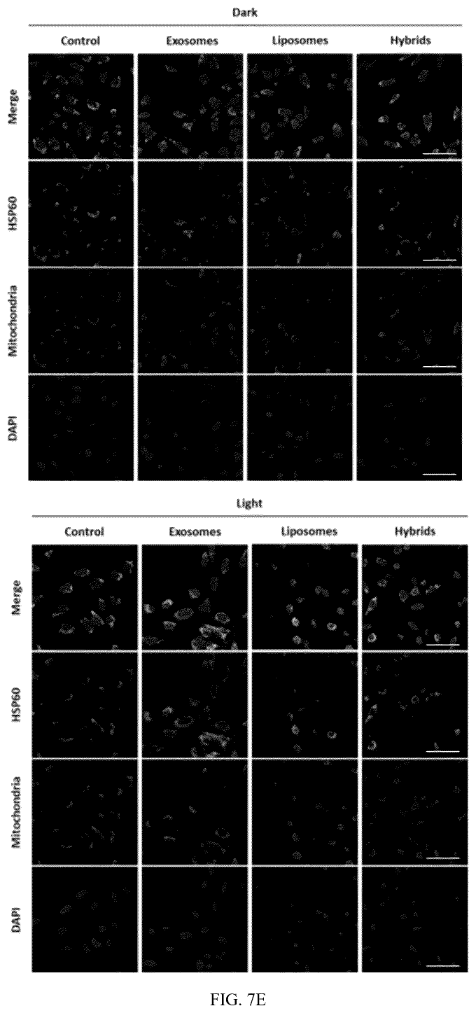

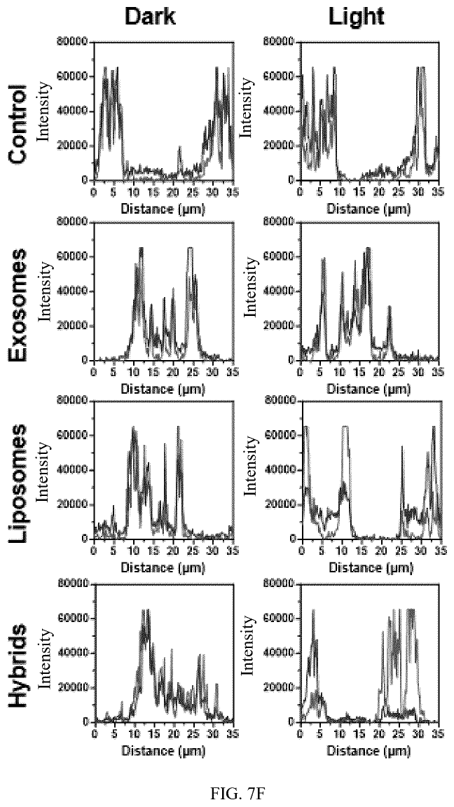

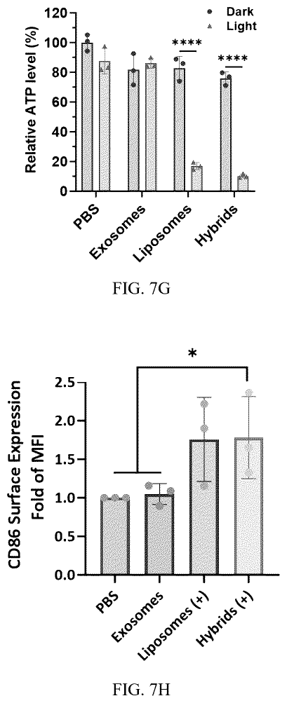

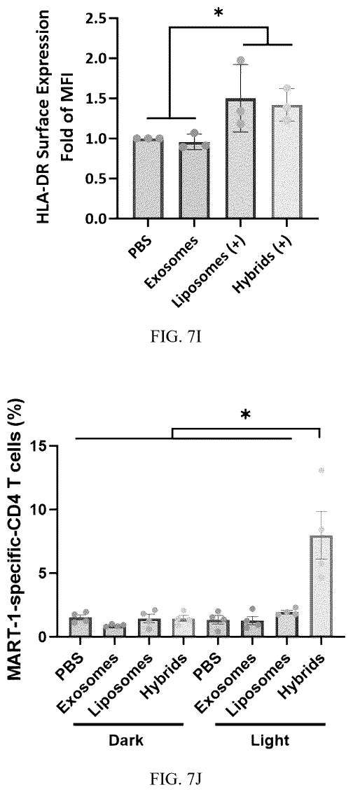

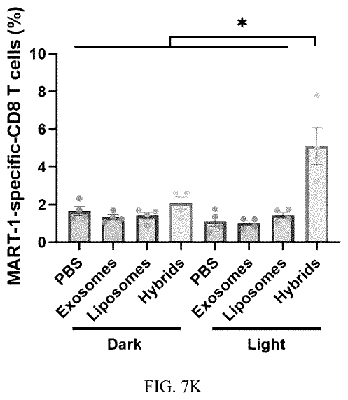

Representative CLSM images show that CRT expression is markedly upregulated on the cell surface of A375 cells after hybrid vesicle treatment with light irradiation (FIGS. 7A and 7B). Concurrently, HMGB1 translocate from the nucleus to the cytoplasm and extracellular milieu under the same conditions (FIGS. 7C and 7D).

Another DAMP marker, mitochondrial heat shock protein 60 (HSP60), is also examined. A375 cells are treated as above, then fixed, permeabilized, blocked, and incubated with FITC anti-HSP60 antibodies and MitoTracker. After PBS washes, cells are stained with DAPI and mounted. Colocalization between HSP60 and mitochondria is analyzed using ZEN 3.3 software. Results indicate significantly reduced colocalization of HSP60 with mitochondria in the irradiated hybrid vesicle group, consistent with DAMP release (FIGS. 7E and 7F).

Intracellular ATP levels—a third ICD indicator—are measured using a luminescence assay (Beyotime S0026). A375 cells in 96-well plates are treated with γδ-T exosomes, hybrid vesicles, or Ce6-loaded liposomes, with or without light irradiation, then harvested after 24 hours for ATP measurement per the kit protocol. The hybrid vesicle group shows decreased intracellular ATP levels compared to controls (FIG. 7G), confirming DAMP release and ICD induction.

Because dendritic cell maturation is critical for eliciting antitumor immune responses, stressed tumor cells are cocultured with human monocyte-derived dendritic cells. CD14+ monocytes are isolated from human PBMCs using Human CD14 Microbeads (Miltenyi Biotec) and differentiated for 7 days in 10% human AB serum RPMI 1640 with GM-CSF (20 ng/ml) and IL-4 (50 ng/ml). Differentiation success is confirmed by CD11c+ frequency via flow cytometry. Differentiated dendritic cells are then cocultured with pretreated A375 cells for 24 hours, and expression of maturation markers CD86 and HLA-DR is analyzed by NovoCyte Quanteon flow cytometer.

After 24 hours, increased CD86 and HLA-DR expression is observed in both hybrid vesicle and liposome groups, indicating promotion of dendritic cell maturation (FIGS. 7H and 7I). To further assess melanoma antigen-specific αβ-T cell responses, human PBMCs are cultured with the stressed tumor cells for 5 days and restimulated with a MART-1 peptide pool covering the entire human MelanA/MART-1 protein. Significantly higher levels of MART-1-specific CD4+ and CD8+ T cell responses are detected in the photo-irradiated hybrid vesicle group (FIG. 7J). Collectively, these results demonstrate that photosensitive hybrid γδ-T vesicles can trigger a light-dependent ICD effect, promote dendritic cell maturation, and induce melanoma antigen-specific CD4+ and CD8+ T cell responses, thereby enhancing antitumor immunity.

### Example 6. In Vivo Anti-Melanoma Growth Effect of Photosensitive Hybrid Vesicles

To investigate the therapeutic efficacy of the hybrid vesicles in vivo, an A375 xenograft tumor-bearing nude mouse model is established. Specifically, 4-6-week-old Balb/c nude mice are subcutaneously implanted with A375 human melanoma cells (1×10⁵ cells per mouse). Ten days after implantation, when the tumor volume reach approximately 100 mm³, the mice are intravenously injected every three days for a total of four doses with either PBS, γδ-T exosomes, Ce6-loaded liposomes, or the hybrid vesicles. The protein amount of γδ-T exosomes matches that in the hybrid vesicles, and the Ce6 concentration of the liposomes is equivalent to that in the hybrid vesicles. In the liposome and hybrid vesicle groups, light irradiation (LED, 650 nm, 100 mW/cm², 10 min) is administered one hour after each injection. Tumor volumes and body weights are monitored daily. According to the Laboratory Animal Unit of the University of Hong Kong regulations, mice bearing tumors ≥17 mm in diameter or experiencing ≥20% body weight loss are euthanized. On day 21, all mice are euthanized, and tumors are excised, weighed, and preserved together with major organs for histological and immunofluorescence analysis. Tumor volume is calculated using the formula: length×[width]²×0.52.

Tumor tissues and major organs (heart, kidney, liver, lung, and spleen) are fixed in 10% formalin, embedded in paraffin, and sectioned. Sections are stained with hematoxylin and eosin (H&E) and imaged using an ECLIPSE Ni-U microscope (Nikon). For immunofluorescence staining, tumor sections are dewaxed in xylene, rehydrated through graded ethanol, and subjected to citrate buffer antigen retrieval. Sections are washed with PBS, permeabilized with 0.3% Triton X-100, and blocked in 10% BSA for 1 hour at room temperature. They are then incubated overnight at 4° C. with PE anti-Ki67 antibodies or for 1 hour at 37° C. with TUNEL agents (Beyotime C1088). After three PBS washes, sections are counterstained with DAPI for 10 minutes, mounted with anti-fade medium, and imaged by CLSM. At the end of the experiment, blood serum is collected, and alanine aminotransferase (ALT) and aspartate transaminase (AST) levels are measured using Solarbio detection kits (BC1550 and BC1560) to evaluate liver function.

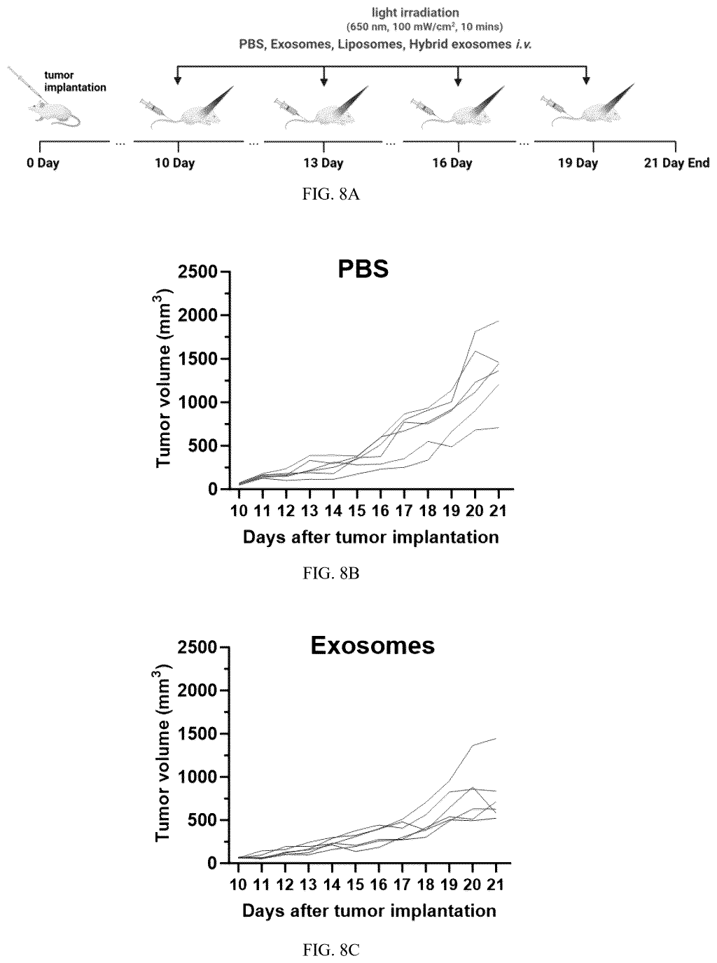

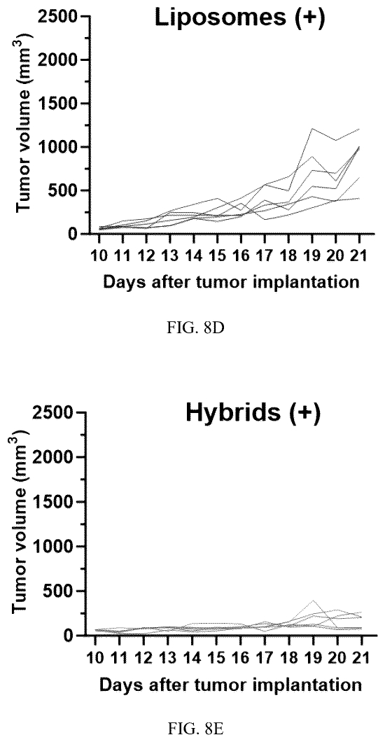

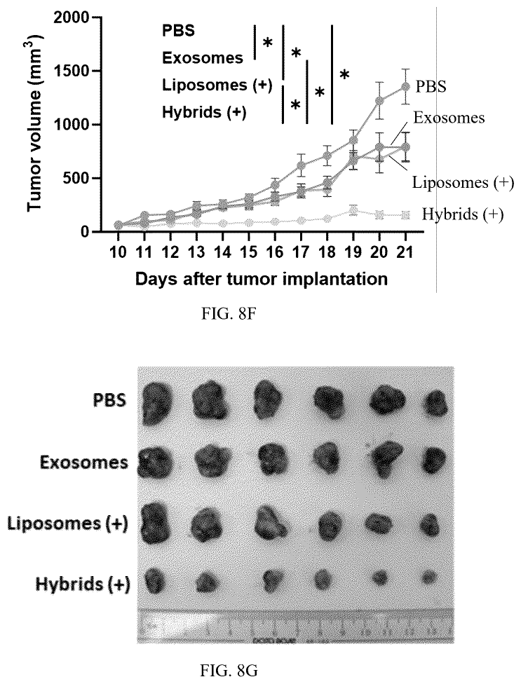

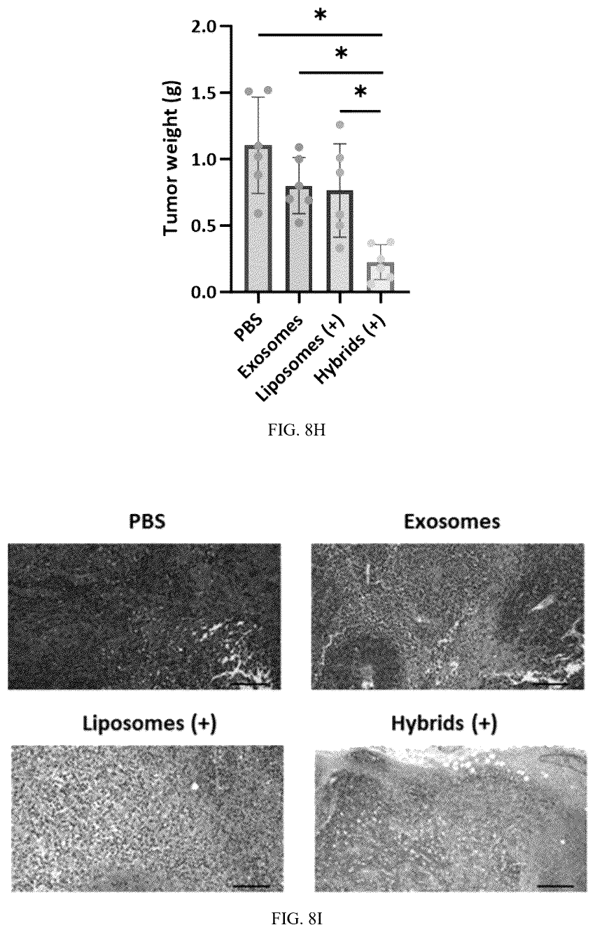

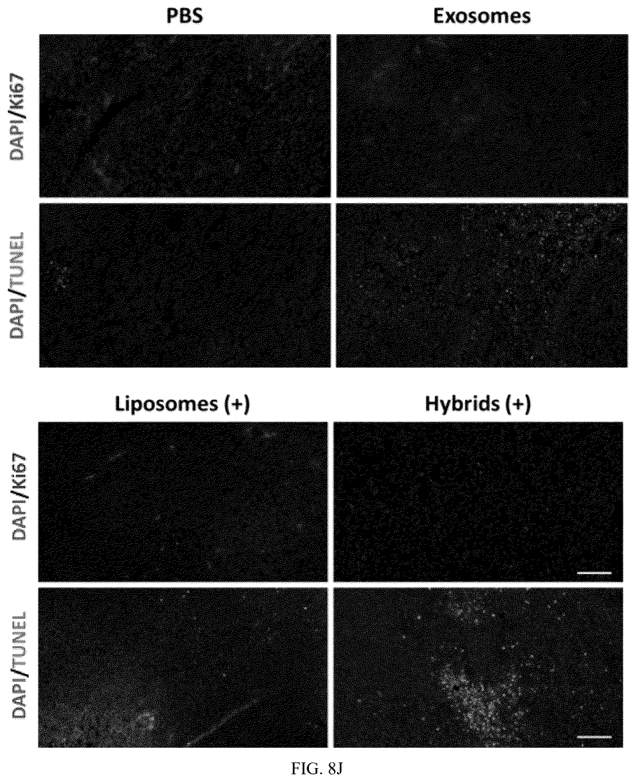

Four groups of mice are monitored over the treatment period, receiving intravenous administrations of the respective formulations every three days starting from day 10 (FIG. 8A). Both γδ-T exosomes and Ce6-loaded liposomes show comparable tumor growth inhibition relative to the control group (FIGS. 8B-8F). Importantly, the hybrid vesicles exhibit a markedly greater tumor-suppressive effect throughout the treatment. At the endpoint, tumor tissues are excised, imaged, and weighed. As shown in FIGS. 8G and 8H, γδ-T exosomes and Ce6-loaded liposomes reduce tumor weight by 28% and 32% compared to the control, whereas hybrid vesicles achieve a significantly greater reduction, highlighting their synergistic antitumor effect. Histological analysis reveals more extensive tumor necrosis in the hybrid vesicle group (FIG. 8I). Immunofluorescence further shows a higher proportion of TUNEL-positive (apoptotic) cells and a lower proportion of Ki67-positive (proliferating) cells in tumors from hybrid vesicle-treated mice compared to other groups (FIG. 8J), indicating enhanced apoptosis and reduced proliferation.

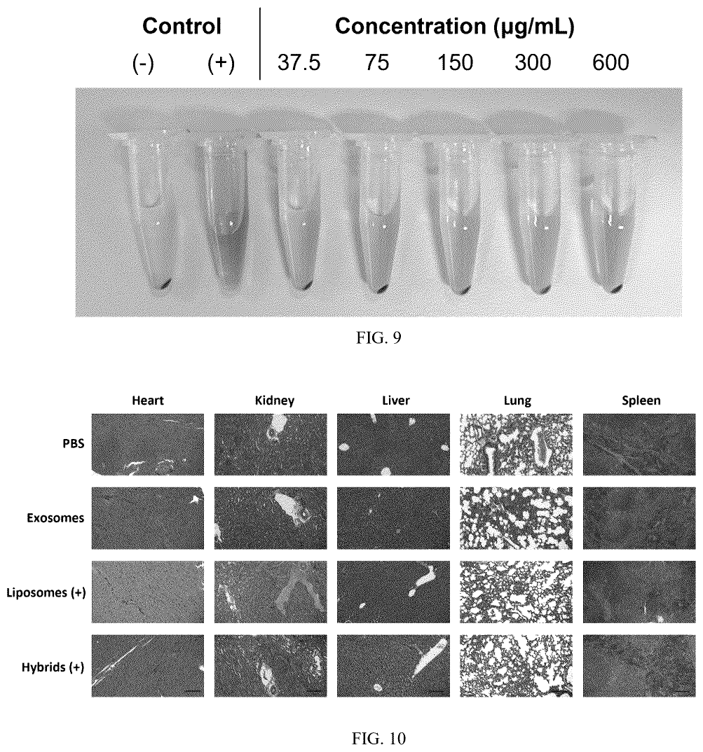

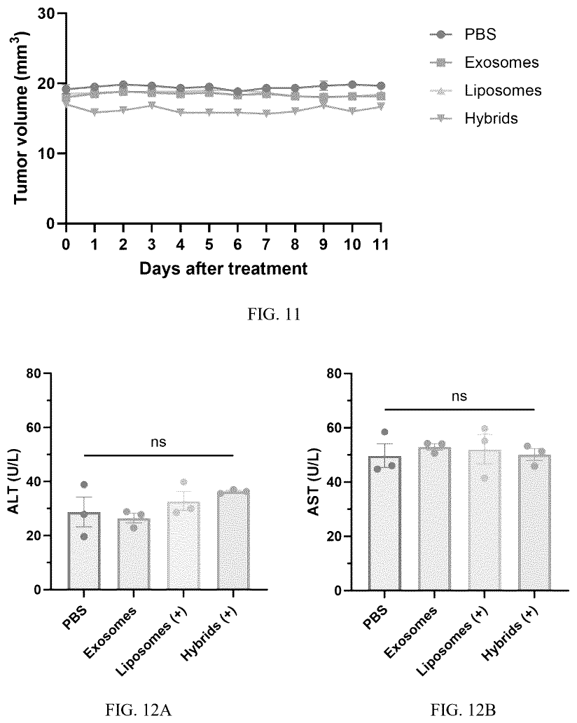

To assess biosafety, a hemolysis assay is performed. Blood from healthy mice is centrifuged at 3,000 rpm for 10 minutes to obtain plasma, which is washed three times with PBS and incubated with gradient concentrations of hybrid vesicles at 37° C. for 3 hours. After centrifugation, plasma pellets are examined, showing that hybrid vesicles have high compatibility with red blood cells during circulation (FIG. 9). Additionally, histological evaluation reveals no significant structural or morphological alterations in heart, liver, spleen, lung, or kidney tissues across all groups (FIG. 10). Body weights remain stable during treatment, suggesting no systemic toxicity (FIG. 11). Furthermore, serum ALT and AST levels show no significant differences among the groups, confirming the excellent biocompatibility and safety of the exosome and liposome formulations (FIG. 12).

In summary, compared with liposomes, the hybrid γδ-T vesicles demonstrate superior active targeting of melanoma cells and higher drug accumulation within tumor tissues, likely mediated by functional surface markers such as PD-1 and CCR5. This approach may also be adapted to deliver other hydrophobic drugs or even hydrophilic cargos using the hybrid γδ-T vesicles platform.

By combining γδ-T exosomes with PDT, the present invention achieves a potent dual-mode antitumor effect. Specifically, the hybrid vesicles simultaneously deliver γδ-T exosome-derived cytolytic molecules—such as granzyme A, granzyme B, perforin, and granulysin—and Ce6-mediated reactive oxygen species (ROS) generation inside melanoma cells. This co-delivery results in a synergistic effect by uniting the advantageous structures of γδ-T exosomes and Ce6 liposomes into a single nanoparticle system. The exosomal membrane facilitates preferential accumulation of Ce6 within melanoma cells, while the photosensitizer-loaded liposomal component enables a strong, light-triggered induction of tumor cell death in combination with γδ-T exosomes. Consistent with reported PDT effects, the hybrid vesicles promote the release of DAMPs from light-irradiated tumor cells, which in turn drives dendritic cell maturation, antigen presentation, and the generation of tumor antigen-specific CD4⁺ and CD8⁺ T-cell responses, thereby achieving photosensitive cancer immunotherapy. Notably, while both Ce6 liposomes and hybrid vesicles can promote dendritic cell maturation, only light-irradiated hybrid vesicles elicit a robust antigen-specific T-cell response. A plausible explanation is that sufficient tumor antigens are released only in the hybrid group after large-scale tumor cell death, enabling a strong, antigen-specific T-cell response. In the animal study, the hybrid vesicles effectively inhibit melanoma growth in an A375 tumor-bearing nude mouse model, inducing marked apoptosis and necrosis within tumor tissues under light irradiation while avoiding obvious side effects in major organs or normal tissues.

The present invention therefore introduces a bioengineered γδ-T exosome platform and a synergistic therapy integrating γδ-T exosome immunotherapy with PDT. This approach provides a novel and efficient strategy for treating superficial carcinomas such as melanoma with high safety and efficacy. Moreover, the invention establishes a practical method for loading hydrophobic drug molecules into γδ-T exosomes at high efficiency while preserving functional surface markers via a membrane fusion process. Building on this platform, other antitumor agents—such as chemotherapeutics or monoclonal antibodies—can likewise be incorporated into biomimetic hybrid nanoparticle formulations for synergistic therapy with γδ-T exosomes. Beyond melanoma, this photoresponsive strategy may be adapted to safely and effectively treat other high-malignancy tumor types, including retinoblastoma, colon cancer, breast cancer, and related indications.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A photosensitive hybrid vesicle, comprising
a photosensitizer-loaded liposome;
and a γδ-T exosome;
wherein the γδ-T exosome and the photosensitizer-loaded liposome are fused together via membrane fusion.

2. The photosensitive hybrid vesicle of claim 1, wherein the photosensitive hybrid vesicle comprises a lipid bilayer structure.

3. The photosensitive hybrid vesicle of claim 1, wherein the photosensitive hybrid vesicle retains functional surface proteins and cytolytic molecules from the γδ-T exosome and incorporates the photosensitizer from the photosensitizer-loaded liposome for generating reactive oxygen species (ROS) upon light irradiation.

4. The photosensitive hybrid vesicle of claim 3, wherein the cytolytic molecules comprise granzyme A, granzyme B, perforin and granulysin; and the functional surface proteins comprise PD-1 and CCR5.

5. The photosensitive hybrid vesicle of claim 1, further comprising a therapeutic agent comprises a chemotherapeutic agent, a monoclonal antibody, an immune checkpoint inhibitor, an immunomodulatory cytokine, an siRNA or miRNA, a gene-editing cargo, a radiotherapeutic agent, a targeted small-molecule inhibitor, and an oncolytic virus.

6. The photosensitive hybrid vesicle of claim 1, wherein the photosensitizer comprises chlorin e6 (Ce6), a porphyrin, phthalocyanine, a bacteriochlorin derivative, or a combination thereof.

7. The photosensitive hybrid vesicle of claim 1, wherein the vesicle is formulated to deliver hydrophobic drugs, hydrophilic drugs, or both simultaneously.

8. The photosensitive hybrid vesicle of claim 1, wherein the membrane fusion is achieved by a freeze-thaw process, polyethylene glycol-mediated fusion, natural incubation, or extrusion-based fusion.

9. The photosensitive hybrid vesicle of claim 1, wherein the vesicle exhibits an average particle size between 50 nm and 300 nm and maintains structural stability under physiological conditions for at least 48 hours.

10. The photosensitive hybrid vesicle of claim 1, wherein the vesicle further comprises targeting ligands or peptides to enhance tissue or tumor specificity.

11. The photosensitive hybrid vesicle of claim 1, wherein the vesicle further comprises one or more targeting ligands selected from antibodies, antibody fragments, peptides, aptamers, or small molecules that specifically bind to a tumor-associated antigen or receptor.

12. A pharmaceutical composition, comprising the photosensitive hybrid vesicle of claim 1 and a pharmaceutically acceptable addition.

13. The pharmaceutical composition of claim 12, wherein the pharmaceutically acceptable addition comprises one or more of an excipient, a stability additive, a carrier, a diluent, or a solubilizer.

14. A method of treating a cancer in a subject, comprising:
administering a therapeutically effective amount of the pharmaceutical composition of claim 12 to the subject; and
exposing a tumor tissue area of the subject to light of a wavelength to activate the photosensitive hybrid vesicle.

15. The method of claim 14, wherein the cancer comprises melanoma, retinoblastoma, colon cancer, breast cancer, gastric cancer, pancreatic cancer, lung cancer, and any solid tumor.

16. The method of claim 14, wherein the wavelength is between 630 nm and 680 nm.

17. The method of claim 14, wherein the pharmaceutical composition is administered intravenously, intratumorally, or topically.

18. A method of manufacturing the photosensitive hybrid vesicle of claim 1, comprising:
preparing a photosensitizer-loaded liposome;
isolating a γδ-T exosome from γδ-T cells;
fusing the photosensitizer-loaded liposome and the γδ-T exosome via a membrane fusion technique; and
obtaining a photosensitive hybrid vesicle;
wherein the photosensitive hybrid vesicle retains functional surface proteins and cytolytic molecules from the γδ-T exosome and incorporates the photosensitizer from the photosensitizer-loaded liposome for generating ROS upon light irradiation.

19. The method of claim 18, wherein the cytolytic molecules comprise granzyme A, granzyme B, perforin and granulysin; and the functional surface proteins comprise PD-1 and CCR5.
